(19) United States
(12) Patent Application Publication
(10) Pub. No.: US 2011/0039799 A1
(43) Pub. Date: Feb. 17, 2011

(54) A1 ADENOSINE RECEPTOR AGONIST POLYMORPHS

(75) Inventors: Ernest Anthony Carra, Foster City, CA (US); Benjamin R. Graetz, San Mateo, CA (US); DeMei Leung, Los Altos, CA (US); Janaki Nyshadham, Fremont, CA (US); Robert Seemayer, Belmont, CA (US); Simon Kwok-Pan Yau, Sunnyvale, CA (US)

Correspondence Address: CV THERAPEUTICS, INC.;Gilead Palo Alto, Inc., 333 Lakeside Drive, Foster City, CA, 94404, US

(73) Assignee: Gilead Palo Alto, Inc., Foster City, CA (US)

(21) Appl. No.: 12/854,023

(22) Filed: Aug. 10, 2010

Related U.S. Application Data
(60) Provisional application No. 61/234,158, filed on Aug. 14, 2009.

(51) Int. Cl. A61K 31/7076; C07H 19/167; A61P 9/00; A61P 3/10; A61P 9/10; A61P 3/04; A61P 25/08; A61P 3/00

(52) U.S. Cl. 514/46; 536/27.3

(57) ABSTRACT

Provided are polymorphs of an A₁ adenosine receptor partial agonist, compositions thereof, methods for their preparation, and methods for their uses.

### CROSS-REFERENCE TO RELATED APPLICATIONS

This application claims priority to U.S. Provisional Patent Application Ser. No. 61/234,158, filed Aug. 14, 2009, the entirety of which of which is incorporated herein by reference.

### FIELD OF THE INVENTION

Provided are polymorphs of an A₁ adenosine receptor agonist, compositions thereof, methods for their preparation, and methods for their use.

### BACKGROUND OF THE INVENTION

Adenosine is a naturally occurring nucleoside, which exerts its biological effects by interacting with a family of adenosine receptors known as A₁, A₂ₐ, A_{2b}, and A₃, all of which modulate important physiological processes.

Stimulation of the A₁ adenosine receptor shortens the duration and decreases the amplitude of the action potential of AV nodal cells, and hence prolongs the refractory period of these cells. Stimulation of A₁ receptors thus provides a method of treating supraventricular tachycardias, including termination of nodal re-entrant tachycardias, and control of ventricular rate during atrial fibrillation and flutter. Acute and chronic disorders of heart rhythm, especially those diseases characterized by rapid heart rate in which the rate is driven by abnormalities in the sinoatrial, atria, and AV nodal tissues, would also benefit from treatment with A₁ adenosine agonists. Such disorders include, but are not limited to, atrial fibrillation and supraventricular tachycardia and atrial flutter. Exposure to A₁ agonists also causes a reduction in the heart rate and a regularization of the abnormal rhythm, thereby improving cardiovascular function.

A₁ agonists, through their ability to inhibit the effects of catecholamines, decrease cellular cAMP, and thus have beneficial effects in the failing heart where increased sympathetic tone increases cellular cAMP levels. The latter condition has been shown to be associated with increased likelihood of ventricular arrhythmias and sudden death. See, for example, B. Lerman and L. Belardinelli Circulation, Vol. 83 (1991), P 1499-1509 and J. C. Shryock and L. Belardinelli, Am. J. Cardiology, Vol. 79 (1997) P 2-10.

A₁ agonists, as a result of their inhibitory action on cyclic AMP generation, have antilipolytic effects in adipocytes that leads to a decreased release of nonesterified fatty acids (NEFA) (E. A. van Schaick et al J. Pharmacokinetics and Biophamaceutics, Vol. 25 (1997) p 673-694 and P. Strong Clinical Science Vol. 84 (1993) p. 663-669). Non-insulin-dependent diabetes mellitus (NIDDM) is characterized by an insulin resistance that results in hyperglycemia. Factors contributing to the observed hyperglycemia are a lack of normal glucose uptake and activation of skeletal muscle glycogen synthase (GS). Elevated levels of NEFA have been shown to inhibit insulin-stimulated glucose uptake and glycogen synthesis (D. Thiebaud et al Metab. Clin. Exp. Vol. 31 (1982) p 1128-1136 and G. Boden et al J. Clin. Invest. Vol. 93 (1994) p 2438-2446). The hypothesis of a glucose fatty acid cycle was proposed by P. J. Randle as early as 1963 (P. J. Randle et al Lancet (1963) p. 785-789). A tenet of this hypothesis would be that limiting the supply of fatty acids to the peripheral tissues should promote carbohydrate utilization (P. Strong et al Clinical Science Vol. 84 (1993) p. 663-669).

The benefit of an A₁ agonist in central nervous disorders has been reviewed (L. J. S. Knutsen and T. F. Murray in Purinergic Approaches in Experimental Therapeutics, Eds. K. A. Jacobson and M. F. Jarvis (1997) Wiley-Liss, N.Y., P-423-470). Briefly, based on experimental models of epilepsy, a mixed A_{2A}:A₁ agonist, metrifudil, has been shown to be a potent anticonvulsant against seizures induced by the inverse benzodiazepine agonist methyl 6,7-dimethoxy-4-ethyl-beta-carboline-3-carboxylate (DMCM, H. Klitgaard Eur. J. Pharmacol. (1993) Vol. 224 p. 221-228). In other studies using CGS 21680, an A_{2A} agonist, it was concluded that the anticonvulsant activity was attributed to activation of the A₁ receptor (G. Zhang et al. Eur. J. Pharmacol. Vol. 255 (1994) p. 239-243). Furthermore, A₁ adenosine selective agonists have been shown to have anticonvulsant activity in the DMCM model (L. J. S. Knutsen In Adenosine and Adenne Nucleotides: From Molecular Biology to Integrative Physiology; eds. L. Belardinelli and A. Pelleg, Kluwer: Boston, 1995, pp 479-487). A second area where an A₁ adenosine agonist has a benefit is in animal models of forebrain ishemia as demonstrated by Knutsen et al (J. Med. Chem. Vol. 42 (1999) p. 3463-3477). The benefit in neuroprotection is believed to be in part due to the inhibition of the release of excitatory amino acids.

Adenosine itself has proven effective in treating disease states related to the A₁ adenosine receptor, for example in terminating paroxysmal supraventricular tachycardia. However, these effects are short-lived because adenosine's half-life is less than 10 seconds. Additionally, as adenosine acts indiscriminately on the A_{2A}, A_{2B}, and the A₃ adenosine receptor subtypes, it also provides direct effects on sympathetic tone, coronary vasodilatation, systemic vasodilatation and mast cell degranulation.

Certain purine nucleosides and their uses as A₁ adenosine receptor agonists are disclosed in U.S. Pat. No. 6,946,449, U.S. Pat. No. 7,005,425, and US 2007/0185051. Each of these references are incorporated herein by reference in their entirety. One of the A₁ adenosine receptor agonists disclosed in these references, 2-{6-[((1R,2R)-2-hydroxycyclopentyl)amino]purin-9-yl}(4S,5S,2R,3R)-5-[(2-fluorophenylthio)methyl]oxolane-3,4-diol (GS-9667), has been chosen for further development, and consequently it is desired to produce this compound in a form that is stable and amenable to large scale synthesis, Several cystalline forms of this compound have been discovered, and surprisingly one polymorh form has been found to be superior with respect to stability to moisture, mechanical stress, pharmaceutical processing, and temperature changes. This polymorph, designated as Form III, has been chosen for future development activities.

### BRIEF SUMMARY OF THE INVENTION

In one aspect, provided are polymorphs (Form III and Form IV) of 2-{6-[((1R,2R)-2-hydroxycyclopentyl)amino]purin-9-yl}(4S,5S,2R,3R)-5-[(2-fluorophenylthio)methyl]oxolane-3,4-diol:

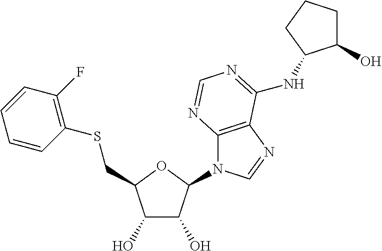

These polymorphs are characterized by a variety of solid state analytical data such as x-ray powder diffraction and differential scanning calorimetry.

In another aspect, provided are compositions comprising a polymorph described herein and a pharmaceutically acceptable carrier.

In other aspects, provided are methods for the preparation of polymorphs described herein, particularly the preparation of Form II from a solution 2-{6-[((1R,2R)-2-hydroxycyclopentyl)amino]purin-9-yl}(4S,5S,2R,3R)-5-[(2-fluorophenylthio)methyl]oxolane-3,4-diol, and the preparation of Form III from Form II.

In other aspects, provided are methods for use of a polymorph described herein to treat a disease in a subject that is alleviated by treatment with an A₁ adenosine receptor agonist, comprising administering to the subject in need thereof a therapeutically effective dose of the polymorph or a composition thereof.

In still other aspects provided are uses of a polymorph described herein in the preparation of a medicament.

These and other aspects of the invention are further described in the Figures and in the Detailed Description that follows.

### BRIEF DESCRIPTION OF THE FIGURES

FIG. 1 shows an XRPD spectrum of polymporph Form III.

FIG. 2 shows a DSC thermogram of polymporph Form III indicating that it is a substantial pure material with an extrapolated onset melting temperature of 147° C. and peak melting temperature of 149° C.

FIG. 3 shows a TGA thermogram of polymporph Form III indicating that Form III is a non-hydrate or non-solvate.

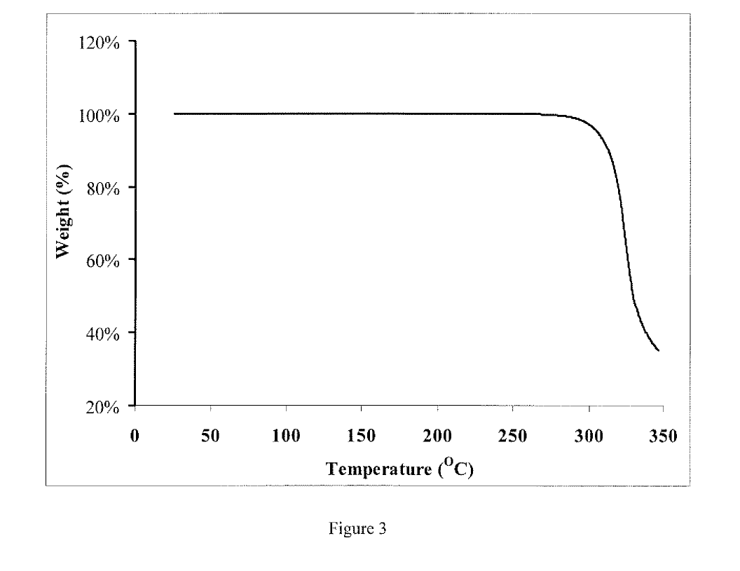

FIG. 4 shows the crystal indexing result of Form III, indicating that Form III is an substantial pure and unique crystalline material.

FIG. 5 shows an XRPD spectrum of polymporph Form IV.

FIG. 6 shows a DSC thermogram of polymporph Form IV indicating that it is a substantial pure material, with an extrapolated onset melting temperature of 117° C. and peak melting temperature of 122° C.

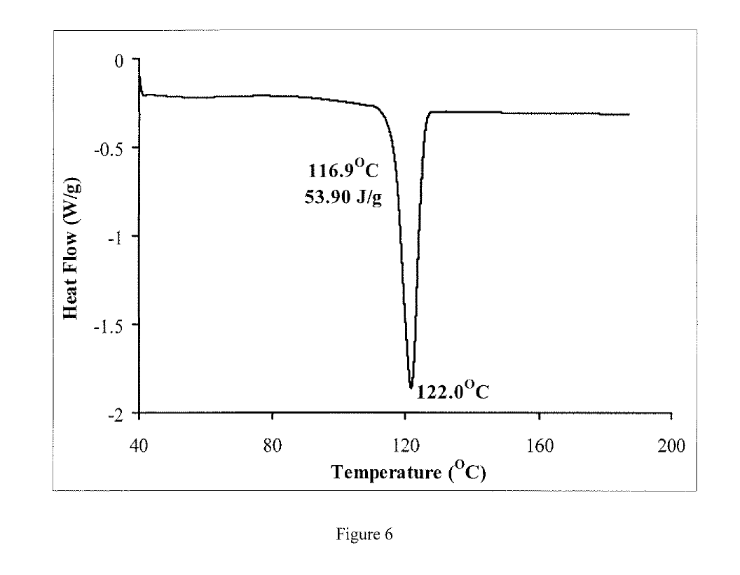

FIG. 7 shows a TGA thermogram of polymporph Form II indicating that Form II is a non-hydrate or non-solvate.

FIG. 8 shows the crystal indexing result of Form IV indicating that Form IV is an substantial pure and unique crystalline material.

FIG. 9 shows the DSC thermogram change in 2-{6-[((1R,2R)-2-hydroxycyclopentyl)amino]purin-9-yl}(4S,5S,2R,3R)-5-[(2-fluorophenylthio)methyl]oxolane-3,4-diol prepared according to Example 1 upon storage at 40° C. and 75% relative humidity for 2 months.

FIG. 10 shows the DSC thermogram of Form III upon storage at ambient temperature and 75% relative humidity for 5 months.

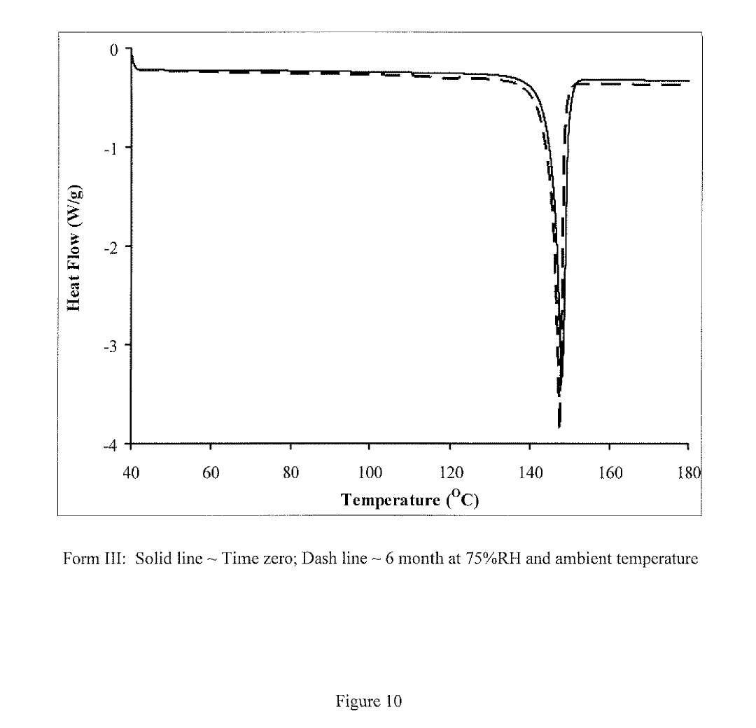

FIG. 11 shows the DSC thermogram of Form IV upon storage at ambient temperature and 75% relative humidity for 3 months.

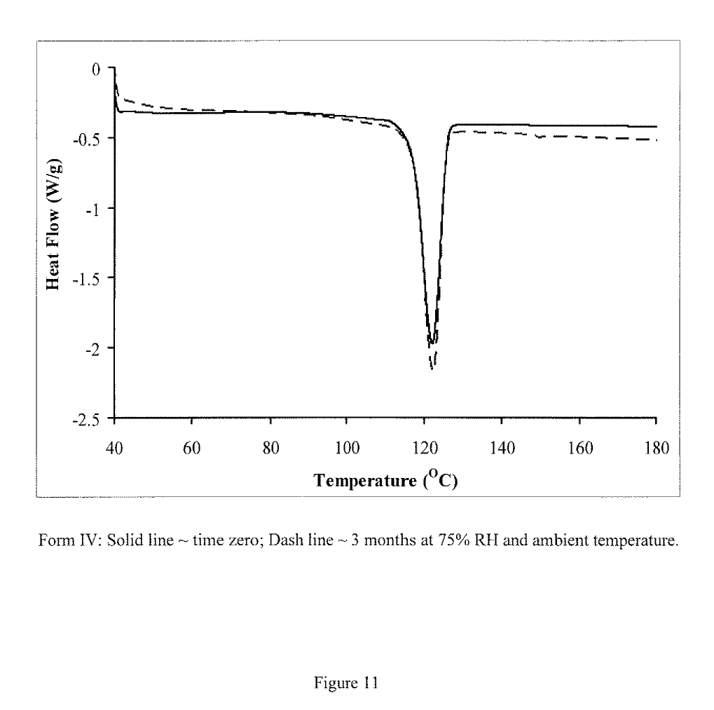

FIG. 12 shows the DSC thermogram change and instability of 2-{6-[((1R,2R)-2-hydroxycyclopentyl)amino]purin-9-yl}(4S,5S,2R,3R)-5-[(2-fluorophenylthio)methyl]oxolane-3,4-diol prepared according to Example 1 when subjected to wet granulation.

FIG. 13 shows solid-state stability by DSC thermogram of Forms III when subjected to wet granulation.

FIG. 14 shows the DSC thermogram change and instability of 2-{6-[((1R,2R)-2-hydroxycyclopentyl)amino]purin-9-yl}(4S,5S,2R,3R)-5-[(2-fluorophenylthio)methyl]oxolane-3,4-diol prepared according to Example 1 when subjected to direct compression and grinding.

FIG. 15 shows solid-state stability by DSC thermogram of Forms III when subjected to direct compression and grinding.

FIG. 16 shows the high hygroscopicity property by the dynamic vapor adsorption of 2-{6-[((1R,2R)-2-hydroxycyclopentyl)amino]purin-9-yl}(4S,5S,2R,3R)-5-[(2-fluorophenylthio)methyl]oxolane-3,4-diol prepared according to Example 1.

FIG. 17 shows the non-hygroscopic property by the dynamic vapor adsorption of Form III.

FIG. 18 shows the non-hygroscopic property by the dynamic vapor adsorption of Form IV.

### DETAILED DESCRIPTION OF THE INVENTION

For purposes of interpreting this specification, the following definitions will apply and whenever appropriate, terms used in the singular will also include the plural and vice versa.

### Abbreviations

XRPD (x-ray powder diffraction); DSC (differential scanning calorimetry); TGA (thermographic analysis data).

As used herein, the term “substantially” refers to degree of variations of +/− by about 1%, about 5%, about 10%, about 15% or about 20%.

As used herein, the term “substantially pure” with respect to a particular polymorphic form of a compound, means that the polymorph form contains about less than 30%, or about less than 20%, or about less than 15%, or about less than 10%, or about less than 5%, or about less than 1% by weight of impurities, such impurities may include other polymorphic forms of the same compound.

As used herein, the term “about” when used in association with a measurement, or used to modify a value, a unit, a constant, or a range of values, refers to variations of +/−3%. A person of ordinary skill in the art would understand that such use of the term “about” does not affect the operation of the invention or its patentability.

As used herein, the term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (See, for example, Remington's Pharmaceutical Sciences, 18th Ed. Mack Printing Company, 1990, pp. 1289-1329, incorporated herein by reference.). Except insofar as any conventional carrier is incompatible with the active ingredient, its use in the therapeutic or pharmaceutical compositions is contemplated.

As used herein, the term “subject” refers to an animal. Preferably, the animal is a mammal. A subject also refers to for example, primate (e.g., human), cow, sheep, goat, horse, dog, cat, rabbit, rat, mouse, fish, bird and the like. In a preferred embodiment, the subject is a human.

The term “therapeutically effective amount” refers to that amount of an active ingredient that is sufficient to effect treatment, as defined below, when administered to a mammal in need of such treatment. The therapeutically effective amount will vary depending upon the subject and disease condition being treated, the weight and age of the subject, the severity of the disease condition, the manner of administration and the like, which can readily be determined by a prescribing physician.

The term “treatment” or “treating” means any treatment of a disease in a mammal, including: (i) preventing the disease, that is, causing the clinical symptoms of the disease not to develop; (ii) inhibiting the disease, that is, arresting the development of clinical symptoms; and/or (iii) relieving the disease, that is, causing the regression of clinical symptoms.

As used herein, the term “agonist” refers to the ability of a compound to interact with a receptor and evoke a maximal effect. This effect is known as the intrinsic efficacy. In contrast, “partial agonists” such as the polymporphs described herein, interact with adenosine A₁ receptors but produce a less than maximal response.

The term “beta-blocker” refers to an agent that binds to a beta-adrenergic receptor and inhibits the effects of beta-adrenergic stimulation. Beta-blockers increase AV nodal conduction. In addition, Beta-blockers decrease heart rate by blocking the effect of norepinephrine on the post synaptic nerve terminal that controls heart rate. Beta blockers also decrease intracellular Ca⁺⁺ overload, which inhibits after-depolarization mediated automaticity. Examples of beta blockers include atenolol, esmolol, sotalol, propranolol, bopindolol, carteolol, oxprenolol, penbutolol, carvedilol, medroxalol, bucindolol, levobunolol, metipranolol, betaxolol, celiprolol, and propafenone.

The term “calcium channel blocker” refers to an agent that blocks voltage-dependent “L-type calcium channel. They are used in treatment of heart diseases, including cardiac arrhythmia, as they have a rate dependent effect upon AV nodal conduction. Examples of calcium channel blockers include amlodipine, bepridil, diltiazem, felodipine, isradipine, nicardipine, nifedipine, nimodipine and verapamil.

The term “cardiac glycoside” refers to a compound with a steroidal nucleus and a lactone ring, and usually has one or more sugar residues. They are used in treatment of heart diseases, including cardiac arrhythmia—they have a rate dependent effect upon AV nodal conduction. Examples of cardiac glycosides include digoxin and digitoxin.

As used herein, “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

Crystalline polymorphic forms of A₁ adenosine receptor partial agonist 2-{6-[((1R,2R)-2-hydroxycyclopentyl)amino]purin-9-yl}(4S,5S,2R,3R)-5-[(2-fluorophenylthio)methyl]oxolane-3,4-diol having the structure shown below are provided herein:

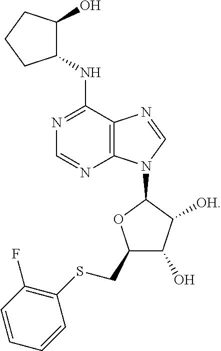

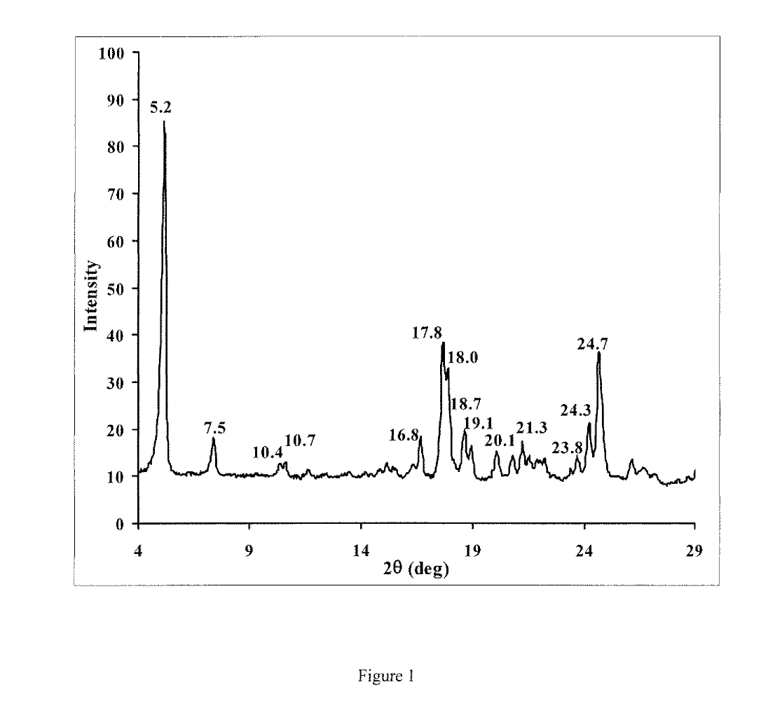

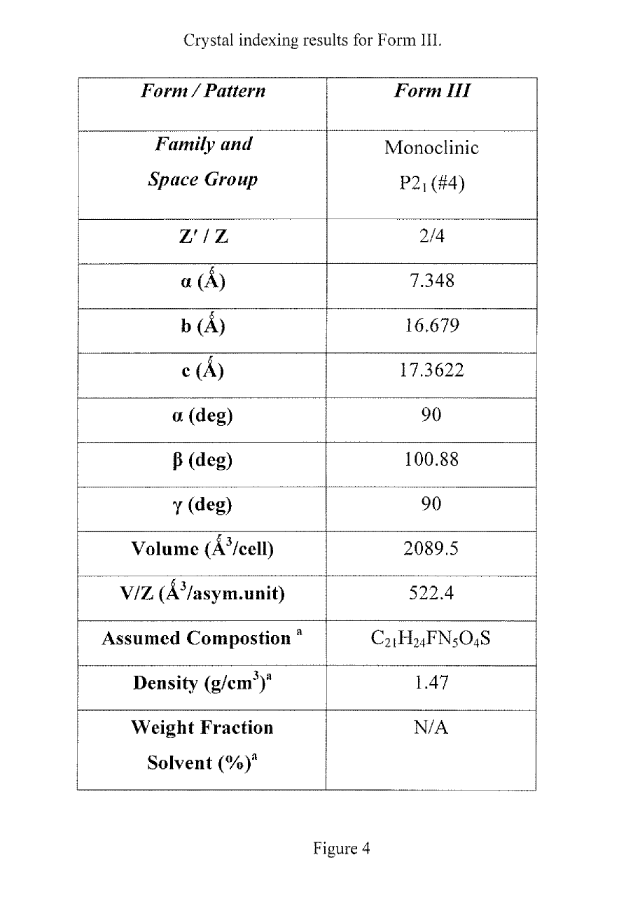

Representative XRPD patterns for Forms III and IV are shown in FIGS. 1 and 4 respectively, and were collected as described in Example 4. Major peaks for each of the Forms are given in Tables I and II below. Relative intensities can vary depending on a number of factors, including sample preparation, mounting, and the instrument and analytical procedure and settings used to obtain the spectrum. Thus the listed peak assignments are intended to encompass variations of plus or minus 0.2 degrees theta.

Thermal data was collected as described in Examples 4 and 5. It is understood that melting point temperatures and thermograms can vary slightly depending on instrumentation and the procedures employed, including the heating rate used. Accordingly, the temperature data and graphs disclosed herein are understood to accommodate such variations.

In one aspect, provided is a polymorph (Form III) of 2-{6-[((1R,2R)-2-hydroxycyclopentyl)amino]purin-9-yl}(4S,5S,2R,3R)-5-[(2-fluorophenylthio)methyl]oxolane-3,4-diol having an X-ray power diffraction pattern comprising characteristic peaks at diffraction angles expressed in degrees 2-theta of about 5.2, 7.5, 16.8, 17.7, 18.0, 18.7, 20.1, 21.3, 24.3 and 24.7. In another aspect, the pattern farther comprises at least one characteristic peak at about 5.2, 7.5 17.7, 18.0 and 24.7. In other aspects, the pattern contains substantially no peaks at 6.3 and 9.5 (i.e. no peaks having an intensity of more than about 2% of the intensity of the strongest peak in the entire pattern).

In one aspect, Form III has substantially the same X-ray powder diffraction pattern as shown in FIG. 1.

In one aspect, Form III is a substantially pure polymorph.

In one aspect, Form III has a DSC extrapolated melting temperature onset of about 147° C. and peak melting temperature of about 149° C.

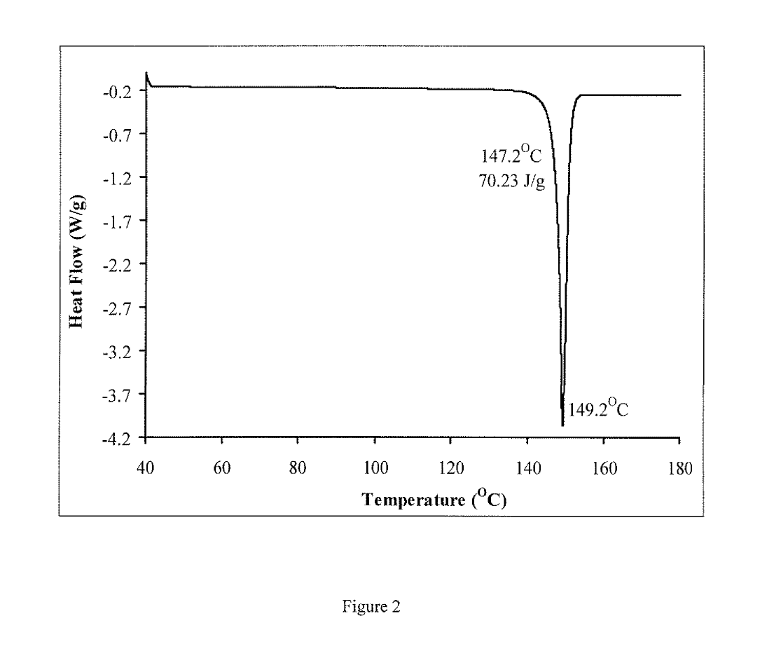

In one aspect, Form III has substantially the same DSC thermogram as shown in FIG. 2.

Form III is prepared according to Example 2 and is a non-hydrate, non-solvate and non-hygroscopic.

In other aspects, provided is a polymormph consisting essentially of Form III and 5%, 4%, 3%, 2%, or 1% of Form IV. Such a polymorph contains no other amorphous solids or crystals other than Forms II and IV.

In one aspect, provided is a polymorph (Form IV) of 2-{6-[((1R,2R)-2-hydroxycyclopentyl)amino]purin-9-yl}(4S,5S,2R,3R)-5-[(2-fluorophenylthio)methyl]oxolane-3,4-diol having an X-ray power diffraction pattern comprising characteristic peaks at diffraction angles expressed in degrees 2-theta of about 6.3, 9.5, 11.4, 12.4, 12.7, 16.4, 17.0, 20.2, 20.5 and 21.6. In another aspect, the pattern further comprises at least one characteristic peak at about 6.3, 9.5, 16.4, 20.2 and 20.5. In other aspects, the pattern contains substantially no peaks at 5.2 and 7.5 (i.e. no peaks having an intensity of more than about 2% of the intensity of the strongest peak in the entire pattern).

In one aspect, Form IV has substantially the same X-ray powder diffraction pattern as shown in FIG. 4.

In one aspect, Form IV is a substantially pure polymorph.

In one aspect, Form IV has a DSC extrapolated melting temperature onset of about 117° C. and peak melting temperature of about 122° C.

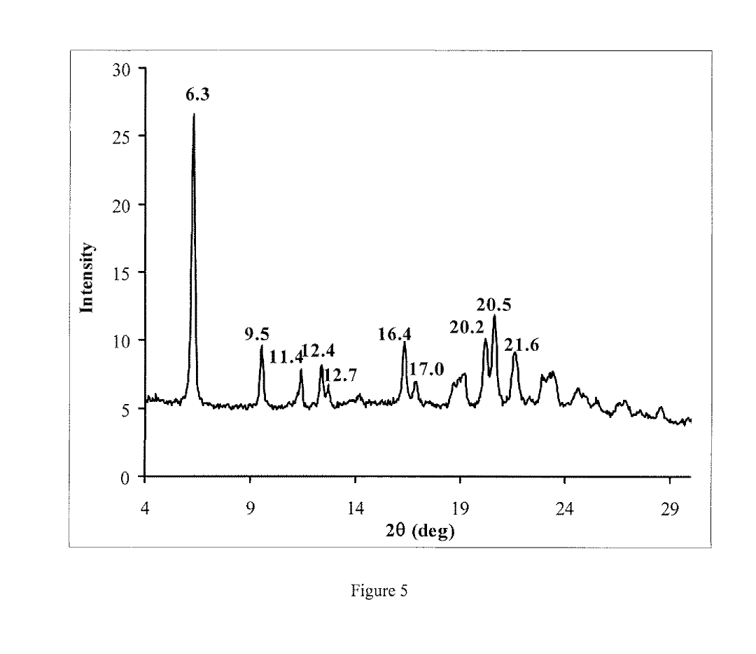

In one aspect, Form IV has substantially the same DSC thermogram as shown in FIG. 5.

Form IV is prepared according to Example 3 and is a non-hydrate and non-hygroscopic.

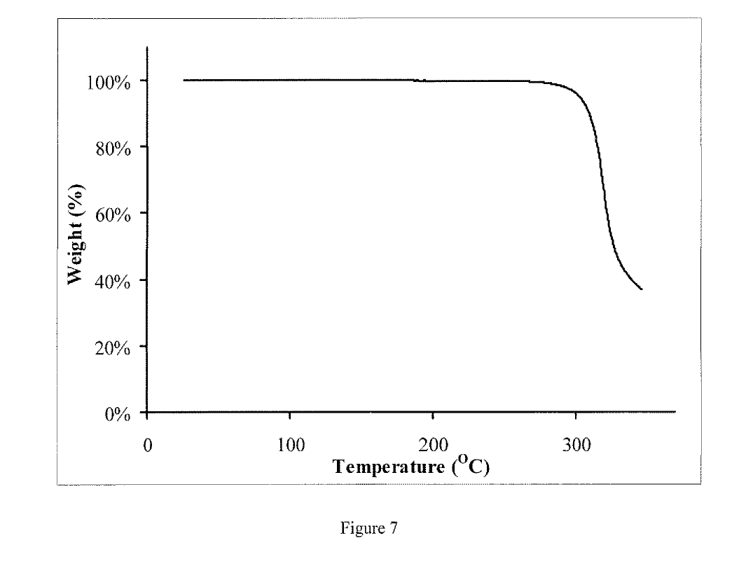

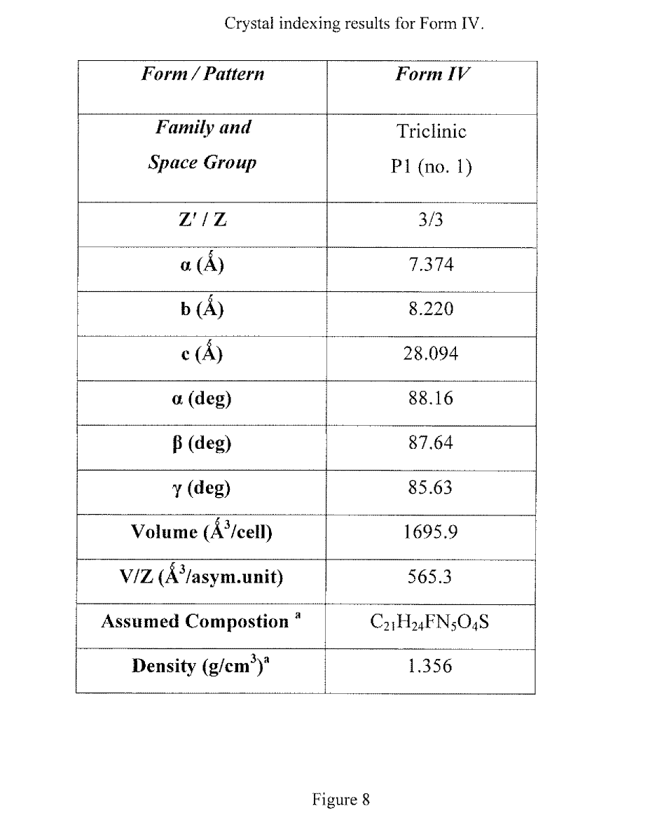

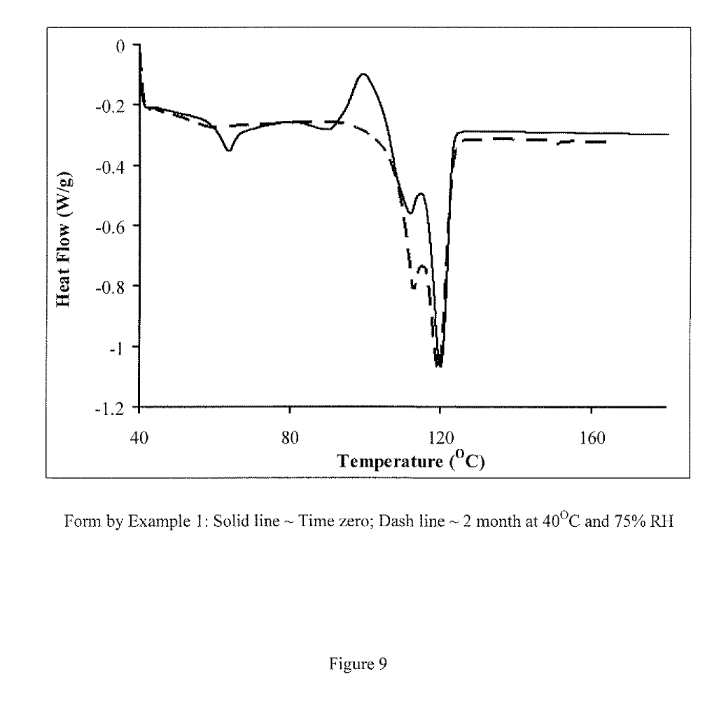

Forms III and IV show enhanced stability in comparision to 2-{6-[((1R,2R)-2-hydroxycyclopentyl)amino]purin-9-yl}(4S,5S,2R,3R)-5-[(2-fluorophenylthio)methyl]oxolane-3,4-diol formed from Example 1 when stored (see FIGS. 7-9).

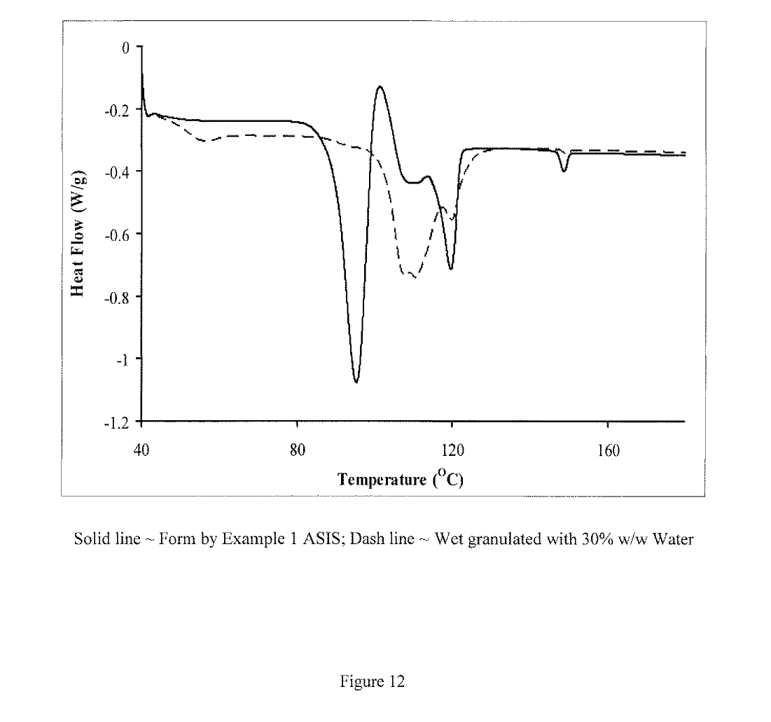

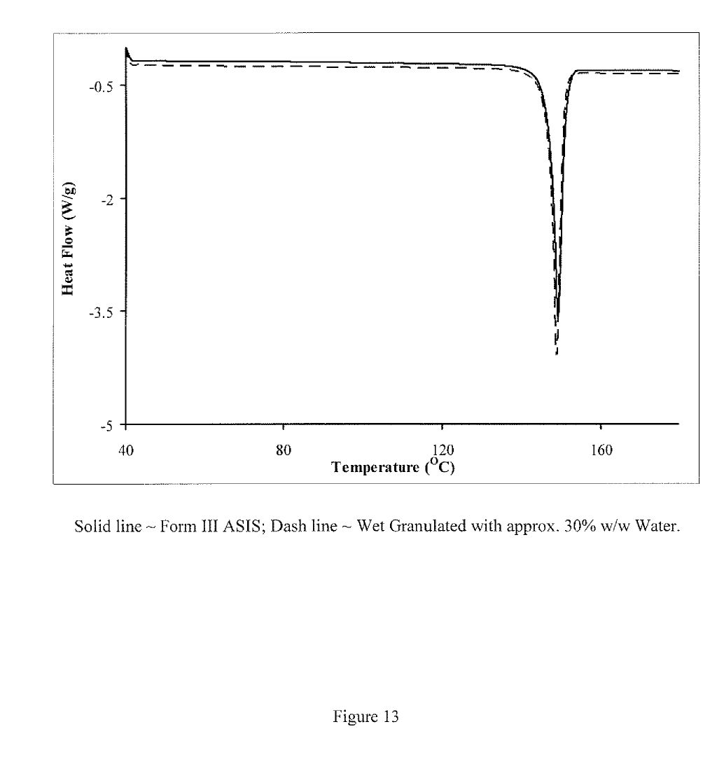

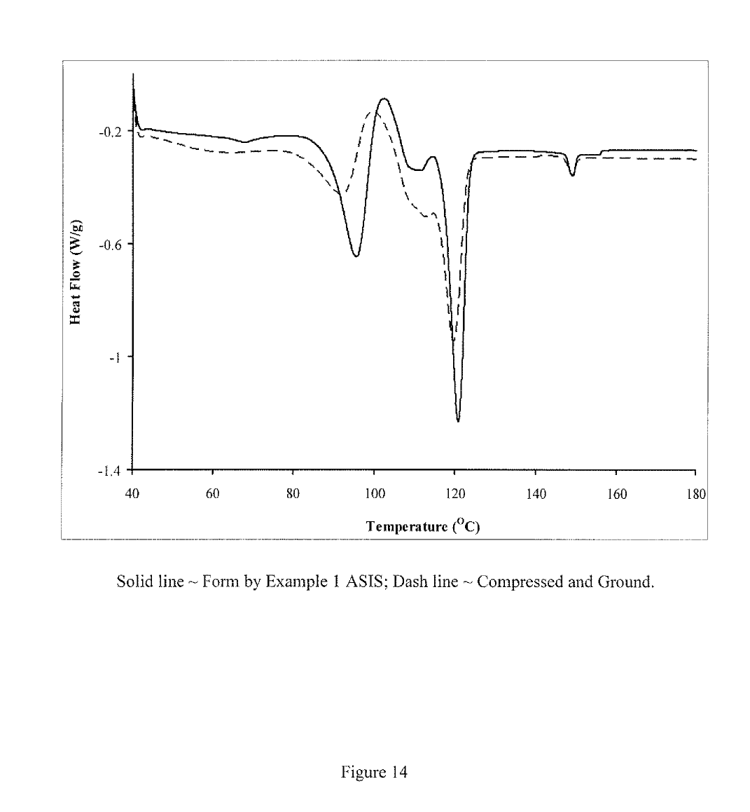

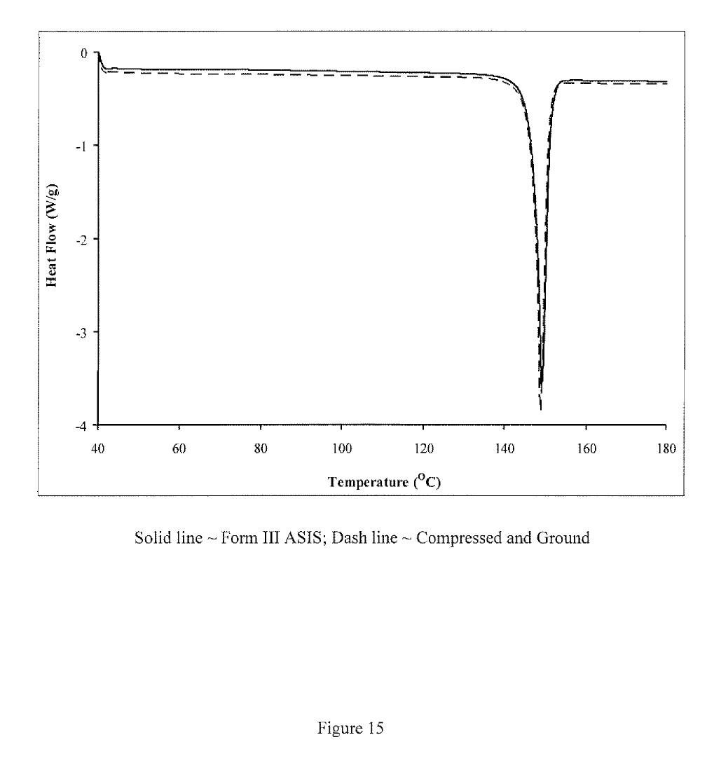

Unlike the compound prepared according to Example 1, Forms III was not affected by wet granulation, compression, or grinding manufacturing processes as shown in FIGS. 12-15. Such characteristics are desirable in formulating and manufacturing medicaments containing this compound.

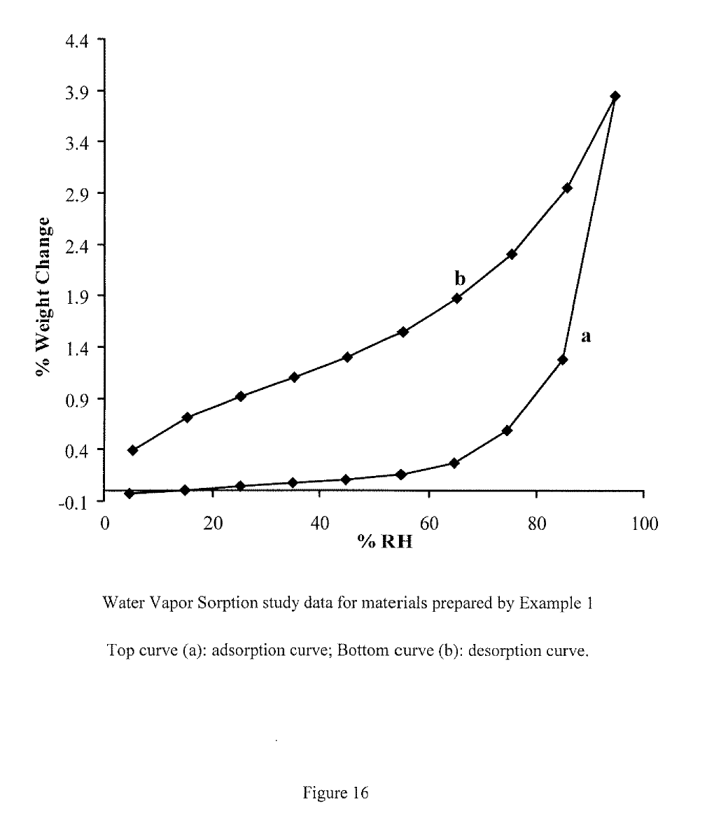

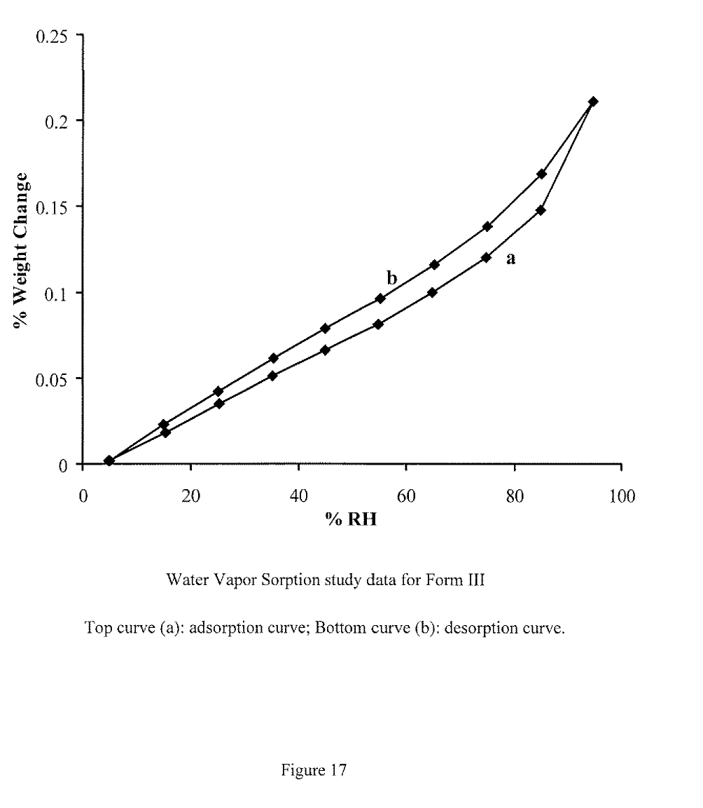

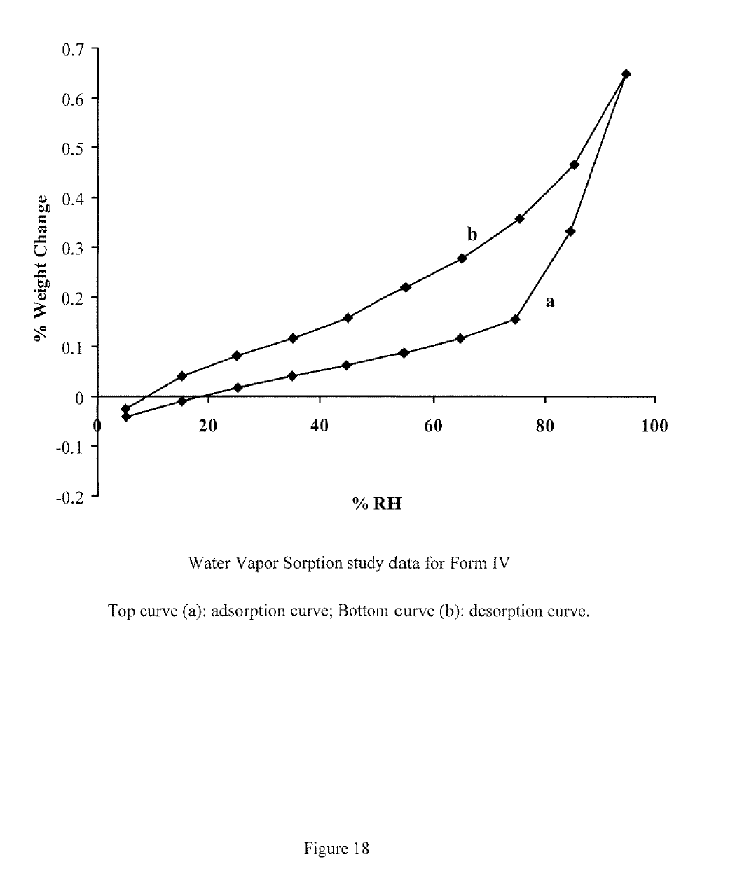

Unlike the compound prepared according to Example 1, Forms III and Form IV were not hygroscopic as shown in FIGS. 16-18. Such characteristics are desirable in manufacturing and storage of medicaments containing this compound

Select XPRD peaks for polymorph Forms III and IV are listed in Tables I and II, respectively.

TABLE I

| | |
|---|---|
| 2 Theta (deg) | Intensity (Counts) |

Form III XPRD Peaks

| | |
|---|---|
| 5.2 | 86 |
| 7.5 | 17 |
| 16.8 | 17 |
| 17.7 | 36 |
| 18.0 | 31 |
| 18.7 | 19 |
| 20.1 | 15 |
| 21.3 | 16 |
| 24.3 | 19 |
| 24.7 | 35 |

Form IV XPRD Peaks

| | |
|---|---|
| 6.3 | 27 |
| 9.5 | 10 |
| 11.4 | 8 |
| 12.4 | 8 |
| 12.7 | 6 |
| 16.4 | 10 |
| 17.0 | 7 |
| 20.2 | 10 |
| 20.5 | 12 |
| 21.6 | 9 |

The polymorphs described herein for use in the treatment of conditions known to respond to administration of a partial or full agonist of an A₁ adenosine receptor. Such conditions include, but are not limited to, acute and chronic disorders of heart rhythm (arrhythmias), especially those diseases characterized by rapid heart rate where the rate is driven by abnormalities in the sinoatrial, atria, and AV nodal tissues. Related disorders include atrial fibrillation, supraventricular tachycardia and atrial flutter, congestive heart failure, stroke, ischemia, stable angina, unstable angina, cardiac transplant, and myocardial infarction. Other conditions include Polycystic Ovarian Syndrome, Stein-Leventhal syndrome, and epilepsy (anticonvulsant activity).

A₁ agonists also have antilipolytic effects in adipocytes that leads to a decreased release of nonesterified fatty acids. US 2007/018505 describes in Example 32 in vivo experiments showing the effects of 2-{6-[((1R,2R)-2-hydroxycyclopentyl)amino]purin-9-yl}(4S,5S,2R,3R)-5-[(2-fluorophenylthio)methyl]oxolane-3,4-diol in lowering circulating free fatty acides (FFA) and serum triglyceride (TG) levels and in improving insulin sensitivity. In one aspect, the polymorphs described herein for the treatment of metabolic diseases such as non-insulin dependent diabetes mellitus, Type II diabetes, Type I diabetes, obesity, and diseases related to decreased glucose tolerance and hyperglycemia.

In other aspects, the provided are use of the polymorphs described herein in combination therapy such as with the administration of a beta blocker, calcium channel blocker, or cardiac glycoside. Non-limiting examples of beta blockers include atenolol, esmolol, sotalol, and propranolol. Non-limiting examples of beta cardiac glycosides include digitalis, digoxin, and digitoxin. Non-limiting examples of calcium channel blockers include amlodipine, bepridil, diltiazem, felodipine, isradipine, nicardipine, nifedipine, nimodipine and verapamil.

### Pharmaceutical Compositions and Administration

When used in combination therapy with additional drugs, the polymorph and one or more drugs and may be administered as a mixture in a single pharmaceutical composition but is preferably administered as two separate pharmaceutical compositions, either concurrently or at different times.

The compositions described herein may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parental, particularly by injection. The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

In making the pharmaceutical compositions that include at least a polymorph as described herein, the active ingredient is usually diluted by an excipient and/or enclosed within such a carrier that can be in the form of a capsule, sachet, paper or other container. When the excipient serves as a diluent, in can be a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The compositions of the invention can be formulated so as to provide quick, sustained, modified, or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The polymorphs described herein are effective over a wide dosage range and is generally administered in a pharmaceutically effective amount. It will be understood, however, that the amount of the polymorph actually administered will be determined by a physician, in the light of the relevant circumstances, including the condition to be treated, the chosen route of administration, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage element, the latter being in the form of an envelope over the former. The two elements can be separated by an enteric layer that serves to resist disintegration in the stomach and permit the inner element to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner.

### EXAMPLES

### Example 1

### Preparation of 2-{6-[((1R,2R)-2-hydroxycyclopentyl)amino]purin-9-yl}(4S,5S,2R,3R)-5-[(2-fluorophenylthio)methyl]oxolane-3,4-diol

2-{6-[((1R,2R)-2-hydroxycyclopentyl)amino]purin-9-yl}(4S,5S,2R,3R)-5-[(2-fluorophenylthio)methyl]oxolane-3,4-diol was prepared as described in U.S. Pat. No. 7,300,923 B2: Example 6, Preparation 2; Example 7, Preparation 2; Example 8, Preparation 2; and Example 9, Preparation 2.

### Example 2

### Preparations of Form III

A—Preparation of Form III Polymorph Using Heat with Stirring Followed by Air Drying

Approximately 10 gram of GS-9667 was mixed in 100 mL of water, which was seeded with 20 mg of Form III polymorph. The mixture was heated at 80° C. for 5 hours with continuous stirring. Form III was obtained by air-drying the mixture for 2-3 days at ambient conditions.

B—Alternative Preparation of Form III Polymorph Using High Heat with Stirring

Approximately 200 milligrams of GS-9667 was dry-heated inside a glass scillation vial on top of a hot-plate for 30 minutes at 138° C. Form III was obtained after the material was allowed to cool under room conditions.

C—Alternative Preparation of Form III Polymorph Using Two Stage Process with Form II Intermediate

Upon completion of the reaction of the final step in the synthesis described in Example 1, the reaction is quenched with water and diluted with ethyl acetate or an other organic solvent such as methyl THF, MTBE, isopropyl acteate, THF, or other polar solvents. After a series of washes and back extraction, a homogenous solution of GS-9667 in 10 to 25 volumes of water saturated ethyl acetate is obtained. The solution is polish filtered and distilled at 0.1 mm Hg to atmospheric pressure to 1 to 15 volumes during which time Form II crystallized. The Form II slurry is cooled, filtered, and the cake dried to not more than 15 weight % residual solvent in the Form II solid. When ethyl acetate is used as the solvent the resisual solvent content must be less than 15 wt %. The form II solid is charged to 10 to 20 volumes of water and the resulting slurry is warmed with stirring at 30° to 80°, preferrably ˜50° C. for several hours depending on temparature during which time Form II converted to Form III. When warmed ot ˜50°, the minimum warming time is ˜3 hours. The slurry is then cooled to no lower than 30° C., filtered, and dried resulting in isolated Form III.

### Example 3

### Preparation of Form IV

10 grams of GS-9667 were suspended in 100 mL xylenes. Under stirring the mixture was heated to reflux (135° C.) over about 1.5 hours. The resulting emulsion was held at reflux temperature for 2 hours and subsequently cooled to room temperature. Form IV was isolated by filtration and dried under oil pump vacuum at 40° C.

### Example 4

### X-Ray Power Diffraction Data Collection

X-ray powder diffraction (XRPD) patterns were collected using an Inel XRG-3000 diffractometer equipped with a curved position sensitive detector with a 2θ range of 120°. Samples were prepared for analysis by packing them into thin-walled glass capillaries. Each capillary was mounted onto a goniometer head and rotated during data acquisition. The monochromator slit was set at 5 mm by 160 μm.

### Example 5

### DSC Collection

Differential scanning calorimetry (DSC) was performed using a TA Instruments differential scanning calorimeter either Q1000 or Q2000. The sample was placed into an aluminum DSC pan, and the weight accurately recorded. The pan was covered with a lid (sometimes the lid was perforated with a (laser) pinhole to allow for pressure release), and then either crimped or hermetically sealed. The sample cell was equilibrated at either 25° C. (Q2000) or 40° C. (Q1000) and heated under a nitrogen purge at a rate of 10° C./min, up to a final temperature of either 180 (Q1000)° C. or 250° C. (Q2000). Indium metal was used as the calibration standard.

### Example 6

### TGA collection

Thermogravimetric (TG) analyses were performed using a TA Instruments 2950 or Q5000 thermogravimetric analyzer. Each sample was placed in an aluminum sample pan and inserted into the TG furnace. The furnace was first equilibrated at 25° C. (2950) or 0° C. (Q5000), then heated under nitrogen at a rate of 10° C./min, up to a final temperature of 350° C. Nickel and Alumel™ were used as the calibration standards

### Example 7

### Water Vapor Sorption Collection

Water vapor sorption data were collected on a VTI SGA-100 Vapor Sorption Analyzer. Adsorption and desorption data were collected over a range of 5% to 95% relative humidity (RH) at 10% RH intervals under a nitrogen purge. The samples were not dried prior to analyses. Equilibrium criteria used for the analyses were less than 0.0100% weight change in 5 minutes, with a maximum equilibration time of 3 hours if the weight criterion was not met. Data were not corrected for the initial moisture content of the samples. NaCl and PVP were used as calibration standards.

### Example 8

### Crystal Indexing

XRPD patterns were collected using a PANalytical X'Pert Pro diffractometer. An incident beam of Cu Kα radiation was produced using an Optix long, fine-focus source. An elliptically graded multilayer mirror was used to focus the Cu Kα X-rays of the source through the specimen and onto the detector. Data were collected and analysed using X′Pert Pro Data Collector software (v. 2.2b). Prior to the analysis, a silicon specimen (NIST SRM 640c) was analyzed to verify the Si 111 peak position. The specimen was sandwiched between 3 μm thick films, analyzed in transmission geometry, and rotated to optimize orientation statistics. A beam-stop was used to minimize the background generated by air scattering. Soller slits were used for the incident and diffracted beams to minimize axial divergence. Diffraction patterns were collected using a scanning position-sensitive detector (X'Celerator) located 240 mm from the specimen.

### Example 9

### Process Instructions for Preparation of Form III

This step has been successfully performed at a maximum scale of 41.4 kg of (2R,3R,4S,5S)-2-(chloromethyl)-5-(6-((1R,2R)-2-hydroxycyclopentylamino)-9H-purin-9-yl)tetrahydrofuran-3,4-diol (GS-454300) input. The (2R,3R,4S,5S)-2-(chloromethyl)-5-(6-((1R,2R)-2-hydroxycyclopentylamino)-9H-purin-9-yl)tetrahydrofuran-3,4-diol was prepared as described in U.S. Pat. No. 7,300,923. The typical times included in the table below are extracted from the largest historical batch.

Process Operation

| | |
|---|---|
| 1. | Charge 1.00 kg of GS-454300, 0.56 kg of potassium carbonate, and 1.5 kg of |
| | dimethylacetamide to a reactor. |
| 2. | Charge 0.38 kg of) 2-fluorothiophenol at such a rate so as to maintain the |
| | temperature below 50° C. (Note 1) |
| 3. | Agitate the mixture at 65° C. (60 to 70° C.) for a period of 2 to 4 h until less than 1 |
| | area % GS-454300 is remaining (monitored by in-process HPLC analysis). (Note 2) |
| 4. | Cool mixture to 20° C. (15 to 25° C.). |
| 5. | Charge 13.5 kg of ethyl acetate to the reaction mixture. |
| 6. | Charge 4 kg of a 5 wt % brine solution and agitate for a minimum of 5 min. (Note 3) |
| 7. | Separate the layers. Hold the aqueous layer for later back extraction. |
| 8. | Charge 4 kg of the brine solution to the ethyl acetate solution and agitate for a |
| | minimum of 5 min.(Note 3) |
| 9. | Separate the layers. Combine the aqueous with the retained aqueous from step 7. |
| 10. | Charge 1.8 kg of ethyl acetate to the combined aqueous solution and agitate for a |
| | minimum of 5 min. |
| 11. | Separate the layers. Discard the aqueous to waste. |
| 12. | Combine the organics and atmospherically distil to ca. 8 L during which time the |
| | product should crystallize. |
| 13. | Cool the slurry at a rate of ca 5° C./h to a final temperature of 0° C. (−5 to 5° C.). |
| 14. | Filter and dry the product under vacuum with a maximum jacket temperature of 70° C. (Note 4) |
| 15. | Charge the dried product to 15 kg of water. |
| 16. | Agitate at 20° C. (15 to 25° C.) for a minimum of 1 h. (Note 5) |
| 17. | Adjust slurry temperature to 60° C. (55 to 65° C.) and agitate at for a minimum of 8 h. |
| 18. | Cool the slurry at a rate of ca 5° C./h to a final temperature of 30° C. (28 to 35° C.). |
| 19. | Filter and dry the product under vacuum with a maximum jacket temperature of 70° C. |

Notes:
1. Exotherm is readily controlled by addition rate and jacket cooling.
2. Typically, NMT 1 A % of GS-454300 is achieved. If necessary, additional 2-fluorothiophenol may be charged.
3. This brine solution is made by charging 0.4 kg of sodium chloride to 7.6 kg of potable water. Agitate until a solution is achieved.
4. The product may be recrystallized at this stage to improve purity. Recrystallization can be from 9 volumes of ethyl acetate/water (97/3) or from 10 volumes of methanol. Both conditions are heated until a solution is achieved then cooled slowly to 0° C. and filtered.
5. Vigorous agitation has been observed to cause foaming on scale.

What is claimed is:

1. A polymorph designated Form III, of 2-{6-[((1R,2R)-2-hydroxycyclopentyl)amino]purin-9-yl}(4S,5S,2R,3R)-5-[(2-fluorophenylthio)methyl]oxolane-3,4-diol: 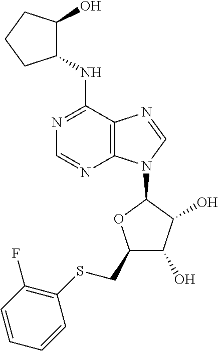
having an X-ray power diffraction pattern substantially the same X-ray powder diffraction pattern as shown in FIG. 1.

2. The polymorph of claim 1, having characteristic peaks at diffraction angles expressed in degrees 2-theta of about 5.2, 7.5, 17.8 and 18.0.

3. The polymorph of claim 1 or 2, wherein the polymorph has a DSC extrapolated melting temperature onset of about 147° C. and peak melting temperature of about 149° C.

4. The polymorph of claim 1, 2, or 3 having substantially the same DSC thermogram as shown in FIG. 2.

5. The polymorph of claims 1 to 4, wherein the polymorph is a substantially pure polymorph.

6. A polymorph (Form II) of 2-{6-[((1R,2R)-2-hydroxycyclopentyl)amino]purin-9-yl}(4S,5S,2R,3R)-5-[(2-fluorophenylthio)methyl]oxolane-3,4-diol: 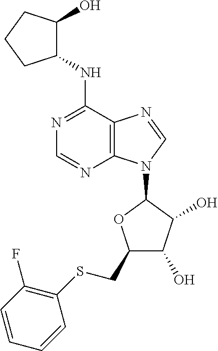
having an X-ray power diffraction pattern substantially the same X-ray powder diffraction pattern as shown in FIG. 4.

7. The polymorph of claim 6, having characteristic peaks at diffraction angles expressed in degrees 2-theta of about 6.3, 9.5, 20.2 and 20.5.

8. The polymorph of claim 6 or 7, wherein the polymorph has a DSC extrapolated melting temperature onset of about 117° C. and peak melting temperature of about 122° C.

9. The polymorph of claim 6, 7, or 8, having substantially the same DSC thermogram as shown in FIG. 5.

10. The polymorph of claims 6 to 9, wherein the polymorph is a substantially pure polymorph.

11. A pharmaceutical composition comprising a polymorph according to any one of claims 1 to 10 and a pharmaceutically acceptable carrier.

12. A method for treating a disease in a subject that is alleviated by treatment with an A₁ adenosine receptor agonist, comprising administering to the subject in need thereof a therapeutically effective dose of the polymorph of any one of claims 1 to 10 or the composition of claim 11.

13. The method of claim 12 wherein the disease is selected from the group consisting of atrial fibrillation, supraventricular tachycardia and atrial flutter, congestive heart failure, antilipolytic effects in adipocytes, Polycystic Ovarian Syndrome, Stein-Levanthal syndrome, decreased glucose tolerance, non-insulin dependent diabetes mellitus, Type II diabetes, Type I diabetes, obesity, epilepsy, stroke, ischemia, stable angina, unstable angina, cardiac transplant, and myocardial infarction.

14. The method of claim 12 in combination with the administration of a beta blocker, calcium channel blocker, or cardiac glycoside.

15. Use of a polymorph of any one of claims 1 to 10 in the preparation of a medicament for the treatment of a disease that is alleviated by treatment with an A₁ adenosine receptor agonist such as atrial fibrillation, supraventricular tachycardia and atrial flutter, congestive heart failure, antilipolytic effects in adipocytes, Polycystic Ovarian Syndrome, Stein-Levanthal syndrome, decreased glucose tolerance, non-insulin dependent diabetes mellitus, Type II diabetes, Type I diabetes, obesity, epilepsy, stroke, ischemia, stable angina, unstable angina, cardiac transplant, and myocardial infarction.

16. Use of claim 15, wherein the medicament is for use in combination with a beta blocker, calcium channel blocker, or cardiac glycoside.

17. A method of producing the polymorph of claim 1, comprising the steps of:
a. preparing the polymorphic Form II of the compound 2-{6-[((1R,2R)-2-hydroxycyclopentyl)amino]purin-9-yl}(4S,5S,2R,3R)-5-[(2-fluorophenylthio)methyl]oxolane-3,4-diol as solid cake having not more than about 15% by weight of organic solvent;
b. mixing the Form II solid in 10-20 volumes of water to form a slurry;
c. heating the slurry to about 30 to 80° C. for at least 2 hours;
d. cooling to no less that 30° C., filtering, and drying the slurry to arrive as isolated Form III.

18. The method of claim 17, wherein Form II of 2-{6-[((1R,2R)-2-hydroxycyclopentyl)amino]purin-9-yl}(4S,5S,2R,3R)-5-[(2-fluorophenylthio)methyl]oxolane-3,4-diol is made by preparing a homogenous solution of GS-9667 in ˜15 volumes of ethyl acetate saturated with water, filtering, and reducing the volume of solvent by distillation at atmospheric pressure to ˜8 volumes, filtering off the solid that crystallized, producing Form II having not more than about 10% by weight of ethyl acetate.

19. The method of claim 17, wherein the slurry is heated in step c to about ˜50° C. for at least 3 hours.
